(19) United States
(12) Patent Application Publication
(10) Pub. No.: US 2018/0245048 A1
(43) Pub. Date: Aug. 30, 2018

(54) METHODS AND COMPOSITIONS FOR EXPANSION AND DIFFERENTIATION OF SKELETAL MUSCLE STEM CELLS OR PROGENITOR CELLS

(71) Applicant: AGENCY FOR SCIENCE, TECHNOLOGY AND RESEARCH, Singapore (SG)

(72) Inventor: Shyh Chang NG, Singapore (SG)

(21) Appl. No.: 15/553,977

(22) PCT Filed: Feb. 25, 2016
(86) PCT No.: PCT/SG2016/050093

§ 371 Date: Aug. 25, 2017

(30) Foreign Application Priority Data
Feb. 25, 2015 (SG) 10201501387X

(51) Int. Cl. C12N 5/077

(52) U.S. Cl. CPC C12N 5/0658; C12N 2501/999; C12N 2500/40; C12N 2501/42; C12N 2501/115

(57) ABSTRACT

The present disclosure describes compositions for preparing a myofiber or myotube from a skeletal muscle stem cell or progenitor cell comprising a carnitine and/or a derivative thereof, a fatty acid a steroid and combinations thereof. Preferred embodiment comprises of 0.1 mM L-carnitine, 0.2 mM 9-cis-linoleic acid and 10 mM dihydrotestosterone. Also disclosed is a composition for inducing expansion of skeletal muscle stem cells or progenitor cells comprising a fibroblast growth factor agonist, a Notch signalling agonist, a nucleic acid, and combinations thereof. Preferred embodiment comprises 20 ng/ml basic fibroblast growth factor (bFGF), 50 μg/ml Delta-like ligand 1 (DLL1), 10 mM hypoxanthine and 1.6 mM thymidine.

### CROSS-REFERENCE TO RELATED APPLICATIONS

This application claims the benefit of priority of Singapore provisional application No. 10201501387X, filed 25 Feb. 2015, the contents of it being hereby incorporated by reference in its entirety for all purposes.

### FIELD OF THE INVENTION

The present invention relates to the fields of cell biology, molecular biology and biotechnology. In particular, the invention relates to differentiating, culturing and expanding skeletal muscle progenitor cells in cell culture.

### BACKGROUND OF THE INVENTION

The use of myogenic stem cells as a cell therapy for various muscle diseases, such as skeletal muscle injuries and cachexia, and for use in regenerative medicine has been attempted for decades, with only moderate success. Myocytes from, for example, induced pluripotent stem cells (iPSCs), are promising candidates for stem cell therapy to regenerate skeletal muscle since they allow allogeneic transplantation. Thus, in order to be able to utilise these myocytes in the large quantities required during therapy, there is an unmet need for a safe and well-tolerated method and/or a composition for expanding and differentiating myoblasts in cell culture.

### SUMMARY

In one aspect, the present invention refers to a composition for preparing a myofiber or myotube from a skeletal muscle stem cell or progenitor cell comprising a carnitine or a derivative thereof, a fatty acid, a steroid and combinations thereof.

In another aspect, the present invention refers to a composition for inducing expansion of skeletal muscle stem cells or progenitor cells comprising a fibroblast growth factor signalling agonist, a Notch signalling agonist, a nucleic acid, and combinations thereof.

In yet another example, the present invention refers to a method for preparing myofibers or myotubes, comprising the step of contacting a skeletal muscle stem cell or progenitor cell with the composition as described herein.

In a further example, the present invention refers to a method for inducing expansion of skeletal muscle progenitor cells comprising the step of contacting a skeletal muscle stem cell or progenitor cell with the composition as described herein.

### BRIEF DESCRIPTION OF THE DRAWINGS

The invention will be better understood with reference to the detailed description when considered in conjunction with the non-limiting examples and the accompanying drawings, in which:

FIG. 1 is a column graph showing the comparison of the level of PAX7 mRNA expression in induced pluripotent stem cell-derived myoblasts treated with 20 ng/ml basic fibroblast growth factor (bFGF), 3 μM CHIR99021, 2.5 μg/ml Delta-like protein 1 (DLL1) and the combination of bFGF/CHIR/DLL1, compared to an untreated control group. The data shown in this figure illustrates that the treatment of the cells with the aforementioned compounds results in an increase in PAX7 expression of the cells, thereby indicating that myoblast function is being promoted, as it is known that PAX7 is a master transcription factor that maintains myoblast identity and proliferation.

FIG. 2 is a heat map depicting the results of the liquid chromatography—mass spectrometry (LC-MS)-based intracellular metabolomics performed on human induced pluripotent stem cells undergoing myogenesis. Embryoid bodies (EB) with drug treatment using bFGF/CHIR/DLL1 are enriched for PAX7 myoblasts, and show higher levels of certain metabolites such as cyclic AMP (cAMP), deoxynucleotides (dNTPs), nucleotides (NTPs) and vitamin B12, relative to EBs with no drug treatment.

FIG. 3 is a heat map showing the levels of OCT3/4, NCAM, AFP and PAX7 mRNA expression in induced pluripotent stem cell-derived myoblasts after screening with 70 different small molecules related to the metabolomics findings, compared to an untreated control group. The data shown in this figure illustrates that the treatment of the cells with compounds such as forskolin, glutamine, hypoxanthine and thymidine (HT) supplement and vitamin B12, can increase PAX7 and the neuromuscular marker NCAM in the cells, thereby indicating increased myoblast function, without increasing the pluripotency marker OCT3/4, or the endoderm marker AFP.

FIG. 4 is a line graph showing a comparison of the population doubling or proliferation rate in myoblasts derived from primary adult human skeletal muscle (hSkM), human embryonic stem cells (hES) or human induced pluripotent stem cells (hiPS), with or without exposure to the combined cocktail of bFGF/CHIR/DLL1 and forskolin, glutamine, hypoxanthine and thymidine (HT) supplement and vitamin B12, dissolved in DMEM media. The data shown in this figure illustrates that the treatment of the cells with the aforementioned cocktail results in a dramatic increase in population doublings or proliferation by at least 2¹²-fold (over 4000-fold), compared to any myoblasts exposed to DMEM (20% FBS) media.

FIG. 5 is a column graph showing the levels of PAX7, MYF5, MYOD1, MYOG, MYHC mRNA expression in myoblasts derived from adult human skeletal muscle (hSkM), human embryonic stem cells (hES) or human induced pluripotent stem cells (hiPS), with or without exposure to the combined cocktail of bFGF/CHIR/DLL1 and forskolin, glutamine, hypoxanthine and thymidine (HT) supplement, and vitamin B12, dissolved in DMEM media. Both passage 6 and passage 1 cells were compared, relative to iPS-derived myoblasts at passage 1 in the cocktail. The data shown in this figure illustrates that the treatment of the cells with the aforementioned cocktail results in an increase in the myoblast markers PAX7 and MYF5, and a decrease in the differentiated myocyte markers MYOG, MYHC over time by passage 6, thereby indicating that the cocktail promotes myoblast proliferation and prevents myoblast differentiation into myocytes. In contrast, DMEM (20% FBS) media decreases the myoblast markers PAX7 and MYF5, and increases the differentiated myocyte markers MYOD1, MYOG, MYHC dramatically within one passage, thereby indicating that regular DMEM media promotes myoblast differentiation.

FIG. 6 is a column graph showing the comparison of the level of PAX7 mRNA expression in induced pluripotent stem cell-derived myoblasts treated with different concentrations of basic fibroblast growth factor (bFGF), Delta-like protein 1 (DLL1), Delta-like protein 4 (DLL4), Jagged protein 1 (JAG1), Jagged protein 2 (JAG2), HT (hypoxanthine and thymidine) supplement, a combination of HT and DLL1, and a combination of HT, DLL1 and bFGF, compared to a vehicle-treated (BSA 0.1%) control group. The data shown in this figure illustrates that the treatment of the cells with the combination of HT 1× (hypoxanthine 10 mM and thymidine 1.6 mM), DLL1 50 μg/ml, and bFGF 20 ng/ml is most optimal for increasing PAX7 expression (˜100-fold) and hence increasing myoblast proliferation.

FIG. 7 is a column graph showing the comparison of the proliferation rate of myoblasts treated with different concentrations of basic fibroblast growth factor (bFGF), Delta-like protein 1 (DLL1), Delta-like protein 4 (DLL4), Jagged protein 1 (JAG1), Jagged protein 2 (JAG2), hypoxanthine and thymidine (HT) supplement, a combination of HT and DLL1, and a combination of HT, DLL1 and bFGF, compared to a vehicle-treated (BSA 0.1%) control group. The data shown in this figure illustrates that the treatment of the cells with the combination of HT 1× (hypoxanthine 10 mM and thymidine 1.6 mM), DLL1 50 μg/ml, and bFGF 20 ng/ml is most optimal for increasing myoblast cell proliferation (˜4000-fold).

FIG. 8 is a heat map depicting the results of the liquid chromatography-mass spectrometry (LC-MS)-based intracellular metabolomics performed on human induced pluripotent stem cells undergoing myogenesis. Monolayers with drug treatment using bFGF/CHIR/DLL1 are enriched for differentiated myotubes, and show higher levels of certain metabolites such as carnitine, fatty acyl-CoA, acetyl-CoA and sterols, relative to monolayers with no drug treatment.

FIG. 9 is a heat map showing the levels of MYOG, MYHC, MYH3, MYH8, MYH7, MYH2, MYH1 expression in induced pluripotent stem cell-derived myotubes after screening with 70 different small molecules related to the metabolomics findings, compared to an untreated control group. The data shown in this figure illustrates that the treatment of the cells with some compounds such as carnitine, linoleic acid, fluvastatin, testosterone, can increase expression of the differentiated myocyte markers MYOG, MYHC, MYH3, MYH8 and adult myotube markers MYH7, MYH2, MYH1, thereby indicating increased efficiency of myoblast differentiation into myotubes.

FIG. 10 is a pair of micrographs showing the morphology and differentiation efficiency of induced pluripotent stem cell-derived myotubes, after exposure to control Dulbecco's modified Eagle's medium (DMEM; 2% horse serum) media or CLFT (carnitine, linoleic acid, fluvastatin, testosterone) supplemented media for 8 weeks. The data shown in this figure illustrates the increased formation of thick multinucleated myotubes upon exposure to the CLFT media.

FIG. 11 is a pair of micrographs showing the morphology and differentiation efficiency of primary human skeletal muscle (hSkM) myoblast-derived myotubes, after exposure to control Dulbecco's modified Eagle's medium (DMEM; 2% horse serum) media or CLFT (carnitine, linoleic acid, fluvastatin, testosterone) media for 3 days. The data shown in this figure illustrates the increased formation of thick multinucleated myotubes upon exposure to the CLFT media.

FIG. 12 is a column graph showing the levels of MYOG, MYHC, MYH3, MYH8, MYH7, MYH2, MYH1 mRNA expression in myotubes derived from primary adult human skeletal muscle (hSkM). The data shown in this figure illustrates that the treatment of the cells with the CLFT media results in an increase in the differentiated adult myotube markers MYOG, MYHC, MYH3, MYH8, MYH7, MYH2, MYH1, thereby indicating that CLFT media enhances primary hSkM myoblast differentiation into adult myotubes.

FIG. 13 is a column graph showing the comparison of the level of adult slow MHC (MYH7) and adult fast MHC (MYH2) mRNA expression in induced pluripotent stem cell-derived myotubes treated with different concentrations of carnitine, O-acetyl-carnitine, 9-cis-linoleic acid, 12-cis-linoleic acid, cis-9-octadecadienoic acid, arachidonic acid, eicosapentaenoic acid, docosahexaenoic acid, linolenic acid, testosterone, estradiol, a combination of 9-cis-linoleic acid (L) and testosterone (T), and a combination of carnitine (C), 9-cis-linoleic acid (L) and testosterone (T), compared to a vehicle-treated (DMSO 0.5%) control group. The data shown in this figure illustrates that the treatment of cells with the CLT combination is most optimal for increasing both adult slow MHC (MYH7) and adult fast MHC (MYH2) expression, and hence increasing adult myotube differentiation.

### DEFINITIONS

As used herein, the term “myogenesis” refers to the generation and formation of muscular tissue, in particular during embryonic development. During the process of myogenesis, mature myocytes are formed through the differentiation and maturation of myoblasts.

As used herein, the term “myoblasts” refers to embryonic (precursor) muscle cells, from which mature contractile cells are derived. These mature contractile cells, commonly known as myocytes, form one of three kinds of muscle cells, which are skeletal myocytes, cardiac myocytes and smooth myocytes, all of which have various properties. The striated cells of cardiac and skeletal muscles are generally referred to as muscle fibres. Cardiomyocytes (that is cardiac myocytes) are the muscle fibres that form the chambers of the heart, and have a single central nucleus. Skeletal muscle fibres (made of fused skeletal myocytes) help support and move the body and tend to have peripheral nuclei. Smooth myocytes control involuntary movements, for example the peristalsis contractions in the oesophagus and stomach.

As used herein, the term “myotube” refers to muscle fibres that are generally formed through the fusion of myoblasts into multi-nucleated fibres.

As used herein, the term “hES” or “human embryonic stem cells” refers to stem cells derived from the undifferentiated inner mass cells of a human embryo. Embryonic stem cells are pluripotent, meaning they are able to grow (i.e. differentiate) into all derivatives of the three primary germ layers: ectoderm, endoderm and mesoderm. In other words, human embryonic stem cells can develop into each of the more than 200 different cell types of an adult human body as long as they are specified to do so. Embryonic stem cells are distinguished by two distinctive properties: their pluripotency, and their ability to replicate indefinitely. Pluripotency distinguishes embryonic stem cells from adult stem cells found in adults. Thus, while embryonic stem cells can generate all cell types in the body, adult stem cells are multipotent and can produce only a limited number of cell types. Additionally, under defined conditions, embryonic stem cells are capable of propagating themselves indefinitely. This allows embryonic stem cells to be employed as useful tools for both research and regenerative medicine, as they can produce limitless numbers of themselves for continued research or clinical use. Because of their plasticity and potentially unlimited capacity for self-renewal, embryonic stem cell therapies have been proposed for regenerative medicine and tissue replacement after injury or disease. Diseases that could potentially be treated by pluripotent stem cells include, but are not limited to, a number of blood and immune-system related genetic diseases, cancers, and disorders; juvenile diabetes; Parkinson's; blindness and spinal cord injuries. Other potential uses of embryonic stem cells include investigation of early human development, the study of genetic disease and as in vitro systems for toxicology testing.

As used herein, the term “primary” in regards to cells in culture, refers to cells that are cultured directly from a subject into cell culture. With the exception of some primary cells derived from tumours, most primary cell cultures have limited lifespan. As opposed to primary cells, an established or immortalized cell line has the acquired ability to proliferate indefinitely, either through random mutation or deliberate modification, such as artificial expression of the telomerase gene.

### DETAILED DESCRIPTION OF THE PRESENT INVENTION

Muscle tissue engineering is one of the important ways for regenerating functionally defective muscles. Muscles, such as, but not limited to, skeletal muscles, are a highly complex and heterogeneous tissue, which serve a multitude of different functions in organism. The process of generating muscle, also known as myogenesis, can be divided into several distinct phases. During embryonic myogenesis, mesoderm-derived structures generate the first muscle fibres of the body, and in subsequent waves additional fibres are generated along these initial template fibres. Attempts to mimic the expansion and differentiation of myoblasts in cell culture is known in the art to be fraught with difficulties, for example the known issue of expanding myoblasts ex vivo without the cells losing their inherent differentiation potential in cell culture due to additives and components required for cell survival and expansion. Therefore, the need for such a method or composition for expanding myoblasts, and their later differentiation, is addressed in the present disclosure.

Using metabolomics and a metabolite/drug screen strategy against both primary human myoblasts (skeletal muscle progenitor cells) and human embryonic stem cell/induced pluripotent stem cell-derived embryoid bodies undergoing myogenic differentiation, a combination of molecules has been identified that can induce both human embryonic stem cell/induced pluripotent stem cell-derived myoblasts and primary human myoblasts to efficiently differentiate into myofibers for human muscle regeneration. Therefore, in one example, the myoblast cell as described herein expresses high levels of Pax7 mRNA as compared to Pax7 mRNA expression in a human embryonic stem cell.

Based on the approach outlined above, a further combination of molecules has been found that is able to further enable expansion of said cells in cell culture. The claimed composition is safe and well tolerated for inducing large-scale expansion of both human embryonic stem cell/induced pluripotent stem cell-derived Pax7⁺ myoblasts and primary human myoblasts, thereby giving rise to the ability to improve and treat a patient's muscle regeneration.

Based on metabolomics analysis of human myoblasts versus human myotubes and a metabolite/drug screen against myoblasts, a composition for efficient differentiation of human myoblasts into myotubes has been found. In contrast, most other metabolites and drugs, which had previously been tried, failed to enhance differentiation of said cells ex vivo. The composition as described herein has been tested on both human embryonic stem cells/induced pluripotent stem cell-derived myoblasts and primary human myoblasts, thereby resulting in a >10-fold higher efficiency in myotube differentiation compared to the current standard treatment for myotube differentiation. Thus, in one example, the present disclosure describes a composition for preparing a myofiber or myotube from a skeletal muscle stem cell or progenitor cell comprising a carnitine or a derivative thereof, a fatty acid, a steroid and combinations thereof.

As used herein, the term “carnitine” refers to a compound derived from an amino acid, which is found in nearly all cells of the body. Carnitine is therefore a generic term for a number of compounds that include, but is not limited to, L-carnitine, acetyl-L-carnitine, and propionyl-L-carnitine. Therefore, in one example, the carnitine or derivative thereof is L-carnitine or an acyl carnitine. In yet another example, the acyl carnitine is O-acetyl-carnitine, O-propionyl-carnitine or O-butanoyl-carnitine. In a further example, the carnitine is L-carnitine.

In one example, the carnitine present in the composition is in a concentration of, but not limited to, between 10 μM to 1 mM, between 5 μM to 0.1 mM, between 0.5 mM to 1 mM, at least 50 μM, at least 100 μM, at least 250 μM, at least 500 μM, about 20 μM, about 70 μM, about 80 μM, about 90 μM, about 95 μM, about 100 μM, about 120 μM, about 180 μM, about 200 μM, about 500 μM, about 800 μM, or about 1000 μM. In one example, the carnitine present in the composition is in a concentration of at least 0.1 mM. In another example, the carnitine present in the composition is in a concentration of about 0.1 mM. In yet another example, the carnitine present in the composition is in a concentration of about 100 μM.

As used herein, the term “fatty acid” refers to a saturated or unsaturated monocarboxylic acid having an aliphatic tail, which may include from about 4 to about 28 carbon atoms. Thus, in one example, the fatty acid is an unsaturated fatty acid. The fatty acid as described herein may be a saturated monocarboxylic acid having the general formula CₙH₂ₙ₊₁COOH, wherein n is a positive integer. In one example, n may be from about 4 to about 28. The aliphatic tail of the fatty acid may be free of hydroxyl functional groups. The fatty acid may occur naturally in the form of esters in fats, waxes, and essential oils or in the form of glycerides in fats and fatty oils. Examples of fatty acids may include, but are not limited to, oleic acid, myristic, palmitic, rumenic, vaccenic, myrisoleic, palmitoleic, alpha-linoleic acid. It may also include any other conventional fatty acids, derivatives thereof, and combinations thereof. In one example, the fatty acid is an omega 3 or an omega 6 fatty acid. In another example, the fatty acid can be, but is not limited to, a linoleic acid, an arachidonic acid, an eicosapentaenoic acid, a docosahexaenoic acid, a linolenic acid and derivatives thereof. In yet another example, the linoleic acid can be, but is not limited to, 9-cis-linoleic acid, 12-cis-linoleic acid, cis-9-octadecadienoic acid and cis-12-octadecadienoic acid. In a further example, the linoleic acid is 9-cis-linoleic acid.

In one example, the fatty acid present in the composition is in a concentration of, but not limited to, between 0.01 mM to 3 mM, between 0.02 to 0.5 mM, between 0.4 mM to 1.8 mM, at least 0.05 mM, at least 0.1 mM, at least 0.15 mM, about 0.1 mM, about 0.18 mM, about 0.2 mM, about 0.5 mM, about 1 mM, about 1.5 mM, about 1.7 mM or about 2 mM. In one example, the fatty acid is present in the composition at a concentration of at least 0.1 mM. In another example, the fatty acid is present in the composition at a concentration of about 0.2 mM.

As used herein, the term “steroid” refers to an organic compound with four rings arranged in a specific configuration. The steroid core structure is composed of seventeen carbon atoms, bonded in four “fused” rings: three six-member cyclohexane rings and one five-member cyclopentane ring. Steroids vary by the functional groups attached to this four-ring core and by the oxidation state of the rings. All steroids are manufactured in cells from the sterols lanosterol (animals and fungi) or cycloartenol (plants). Lanosterol and cycloartenol are derived from the cyclization of the triterpene squalene. In humans, steroid hormones, such as sex hormones include, but not limited to, estrogen, progesterone, androgen, testosterone, dehydroepiandrosterone, androstenedione, dihydrotestosterone, aldosterone, estradiol, estrone, estriol, cortisol, calcitriol, calcidiol and derivatives and analogues thereof. The term steroid, as used herein, encompasses both natural and synthetic steroids and derivatives and analogues thereof. Thus, in one example, the steroid is a sex steroid. In another example, the sex steroid is either an androgen or estrogen. In yet another example, the steroid is a testosterone, estradiol or a derivative thereof. In a further example, the steroid is dihydrotestosterone.

In one example, the steroid present in the composition is in a concentration of, but not limited to, between 0.5 nM to 150 nM, between 1 nM to 100 nM, between 50 nM to 75 nM, at least 0.8 nM, at least 1.6 nM, at least 8 nM, about 3 nM, about 5 nM, about 10 nM, about 11 nM, about 15 nM, about 20 nM, about 40 nM, about 60 nM or about 80 nM. In one example, the steroid is present in the composition in a concentration of at least 10 nM. In one example, the steroid is present in a concentration of about 10 nM.

The composition as described herein may include the further optional component of a statin. As used herein, the term “statin”, also known as HMG-CoA reductase inhibitors, refers to a class of cholesterol lowering compounds and/or molecules that inhibit the enzyme HMG-CoA reductase which plays a central role in the production of cholesterol. High cholesterol levels have been associated with cardiovascular disease (CVD). Statins are also known to promote the production of low-density lipoprotein (LDL)-binding receptors in the liver resulting in a usually marked decrease in the level of LDL and a modest increase in the level of high-density lipoprotein (HDL) circulating in blood plasma. Examples of a statin are, but are not limited to, type 1 or type 2 statins, which can include, but are not limited to fluvastatin, lovastatin, simvastatin, pravastatin, atorvastatin, rosuvastatin, pitavastatin, cerivastatin, mevastatin, and derivatives thereof. In one example, the statin is a type 1 or type 2 statin. In another example, the statin is, but is not limited to fluvastatin, lovastatin, simvastatin, pravastatin, atorvastatin, and derivatives thereof.

As used herein, the term “progenitor cell” refers to a biological cell that, similar to a stem cell, has the ability to differentiate into a specific type of cell, but is already more specific than a stem cell (that is it is further down the path of differentiation than a true stem cell) and is pushed to differentiate into its “target” cell. A progenitor cell may also be described as being oligopotent or unipotent. The most important difference between stem cells and progenitor cells is that stem cells can replicate indefinitely, whereas progenitor cells can divide only a limited number of times. Examples of progenitors found in a human are, for example, but not limited to, satellite cells found in muscles, bone marrow stromal cells found within basal cell of epidermis, pancreatic progenitor cells, angioblasts or endothelial progenitor cells, and blast cells. Therefore, in one example, the skeletal muscle progenitor cell is a myosatellite cell. In another example, the skeletal muscle stem cell is a myoblast cell.

The present disclosure refers to the use of a composition for the differentiation of myoblasts into, for example, myotubes. In one example, the composition as described herein comprises a carnitine, a fatty acid, and a steroid. In another example, the composition as described herein comprises L-carnitine, a fatty acid and a steroid. In another example, the composition comprises a carnitine, linoleic acid, and a steroid. In yet another example, the composition as described herein comprises a carnitine, a fatty acid and testosterone. In a further example, the composition comprises L-carnitine, a fatty acid and testosterone. In another example, the composition as described herein comprises a carnitine, linoleic acid and testosterone. In a further example, the composition as described herein comprises L-carnitine, linoleic acid and testosterone. In yet another example, the composition as described herein comprises L-carnitine in a concentration of about 0.1 mM, linoleic acid in a concentration of about 0.2 mM and testosterone in a concentration of about 10 nM.

Another source of cells with differentiation potential are, for example, induced pluripotent stem cells. As used herein, the term “induced pluripotent stem cell” refers to a type of pluripotent stem cell that can be generated directly from adult cells. Because these pluripotent stem cells can propagate indefinitely, as well as give rise to various other cell type in the body, such as, but not limited to, neurons, heart, pancreatic, and liver cells, these pluripotent cells represent a single source of cells that could be used to replace those lost to damage or disease. Since induced pluripotent stem cells can be derived directly from adult tissues, they not only bypass the need for embryos, from which pluripotent stem cells can be isolated, but can also be made in a patient-matched manner, which means that each individual can have their own pluripotent stem cell line, without the risk of immune rejection associated with implants or foreign tissue. Thus, in one example, the myoblast cell is derived from an embryonic stem (ES) cell, an induced pluripotent stem (IPS) cell, a mesenchymal stem cell, a neural stem cell or a multipotent stem cell. In another example, the myoblast cell is a primary myoblast cell. In yet another example, the skeletal muscle stem cell or progenitor cell is derived from a mammal. In another example, the skeletal muscle stem cell or progenitor cell is derived from a human, rodent or primate.

Furthermore, based on metabolomics analysis of human myoblasts and a metabolite/drug screen against myoblasts, a method, as well as a composition, for large-scale expansion of human myoblasts has been found. It had been previously suggested that fibroblast growth factor (FGF) and Wnt signalling play an important role in embryonic myogenesis. Based on this, the present disclosure combines a fibroblast growth factor (basic fibroblast growth factor; bFGF) and a Wnt signalling agonists (CHIR99201) with a Notch agonist (DLL1) with other components based on the metabolic finding, e.g. forskolin (a cAMP agonist), glutamine, hypoxanthine, thymidine, and cobalamin. In contrast, most other metabolites and drugs that had been previously tested failed to expand myoblasts in culture. The composition as claimed herein has been tried and tested on both human embryonic stem cells/induced pluripotent stem cell-derived myoblasts and human primary myoblasts, resulting in a 4000-fold expansion in cell culture. The further composition described herein enables and induces large-scale expansion of both human embryonic stem cells and induced pluripotent stem cell-derived Pax7⁺ myoblasts for use in, for example, high-throughput drug screens, disease modelling and myoblast transplantation. Thus, in one example, the present disclosure describes a composition for inducing expansion of skeletal muscle stem cells or progenitor cells comprising a fibroblast growth factor signalling agonist, a Notch signalling agonist, a nucleic acid, and combinations thereof.

As used herein, the term “fibroblast growth factor” refers to a family of growth factors, whose members are known to be involved in, but not limited to, angiogenesis, wound healing, embryonic development and various endocrine signalling pathways. The fibroblast growth factors (FGFs) are usually heparin-binding proteins and interaction with cell-surface-associated heparan sulfate proteoglycans, the interaction of which has been shown to be essential for fibroblast growth factor signal transduction. Fibroblast growth factors are known to be key players in the processes of proliferation and differentiation of wide variety of cells and tissues. As used herein, the term “agonist” refers to a molecule (of chemical, synthetic or natural origin) that binds to a receptor and activates the receptor to produce a biological response. Whereas an agonist causes an action, an antagonist blocks the action of the agonist. An inverse agonist causes an action opposite to that of the agonist. Therefore, a “fibroblast growth factor agonist” refers to a molecule that binds to the same receptors and that elicits the same biological reaction as a fibroblast growth factor. Thus, in one example, the fibroblast growth factor signalling agonist is a fibroblast growth factor (FGF). In another example, the fibroblast growth factor signalling agonist can be, but is not limited to, FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22 and FGF23. In yet another example, the fibroblast growth factor signalling receptor agonist is FGF2 (basic fibroblast growth factor; bFGF) or a derivative thereof. In a further example, the fibroblast growth factor signalling receptor agonist is FGF2 (basic fibroblast growth factor; bFGF).

In one example, the fibroblast growth factor agonist present in the composition is in a concentration of, but not limited to, between 1 ng/ml to 250 ng/ml, between 100 ng/ml to 200 ng/ml, between 15 ng/ml to 35 ng/ml, at least 5 ng/ml, at least 18 ng/ml, at least 25 ng/ml, at least 45 ng/ml, about 2 ng/ml, about 10 ng/ml, about 18 ng/ml, about 23 ng/ml, about 35 ng/ml, about 50 ng/ml or about 150 ng/ml. In one example, the fibroblast growth factor agonist is present in the composition in a concentration of at least 20 ng/ml. In another example, the fibroblast growth factor agonist is present in the composition in a concentration of about 20 ng/ml.

As used herein, the term “Notch signalling agonist” refers to a molecule that acts as an agonist for the Notch signalling pathway. Notch signalling is an evolutionarily conserved pathway in multicellular organisms that regulates cell-fate determination during development and maintains adult tissue homeostasis. The Notch pathway mediates juxtacrine cellular signalling, wherein both the signal sending and receiving cells are affected through ligand-receptor crosstalk by which an array of cell fate decisions in neuronal, cardiac, immune, and endocrine development are regulated. Notch receptors are usually, but not limited to, single-pass transmembrane proteins composed of functional extracellular (NECD), transmembrane (TM), and intracellular (NICD) domains. Notch receptors are activated via ligand binding in a manner regulated by Deltex and inhibited by NUMB. In mammalian signal-sending cells, members of the Delta-like (DLL1, DLL3, DLL4) and the Jagged (JAG1, JAG2) families serve as example of binding ligands for Notch signalling receptors. Thus, in one example, the Notch signalling agonist is a Delta-like ligand (DLL), a Jagged/Serrate ligand or a derivative thereof. In another example, the Delta-like ligand (DLL) is selected from a group consisting of Delta-like 1 (DLL1), Delta-like 3 (DLL3) and Delta-like 4 (DLL4). In yet another example, the Delta-like ligand (DLL) is Delta-like 1. In yet another example, the Jagged/Serrate ligand is selected from a group consisting of Jagged 1 (JAG1), Jagged 2 (JAG2) and Serrate.

In one example, the Notch signalling agonist present in the composition is in a concentration of, but not limited to, between 0.1 μg/ml to 80 μg/ml, between 5 μg/ml to 20 μg/ml, between 15 to 60 μg/ml, between 40 to 78 μg/ml, at least 1 μg/ml, at least 10 μg/ml, at least 20 μg/ml, at least 30 μg/ml, at least 40 μg/ml, at least 50 μg/ml, at least 60 μg/ml, about 8 μg/ml, about 18 μg/ml, about 26 μg/ml, about 35 μg/ml, about 45, μg/ml, about 48 μg/ml, about 50 μg/ml, about 55 μg/ml, about 64 μg/ml, or about 75 μg/ml. In one example, the Notch signalling agonist is present in the composition in a concentration of at least 45 μg/ml. In one example, the Notch signalling agonist is present in the composition in a concentration of about 50 μg/ml.

As used herein, the term “nucleic acid” refers to biopolymers, or large biomolecules, essential for all known forms of life. Nucleic acids, which include, but are not limited to DNA (deoxyribonucleic acid) and RNA (ribonucleic acid), are made from monomers known as nucleotides. The basic component of biological nucleic acids is the nucleotide, each of which contains a pentose sugar (ribose or deoxyribose), a phosphate group, and a nucleobase. If the sugar is deoxyribose, the polymer is DNA (deoxyribonucleic acid). If the sugar is ribose, the polymer is RNA (ribonucleic acid). When all three components are combined, they form a nucleic acid. Nucleotides are also known as phosphate nucleotides. As used herein, the term “nucleobase” refers to nitrogen-containing biological compounds (nitrogenous bases) found linked to a sugar within nucleosides—the basic building blocks of deoxyribonucleic acid (DNA) and ribonucleic acid (RNA). Often simply called bases in genetics, their ability to form base pairs and to stack upon one another lead directly to the helical structure of DNA and RNA. Thus, in one example, the nucleic acid is derived from a nitrogenous base selected from a group consisting of hypoxanthine, adenine, guanine, thymine, cytosine, inosine, xanthine, a derivative of the aforementioned nitrogenous bases and combinations thereof. In another example, the composition as described herein comprises at least one, at least two, one, two, three four or more nucleic acids. In yet another example, the composition comprises two nucleic acids.

In one example, the nucleic acid present in the composition is in a concentration of, but not limited to, between 0.1 mM to 15 mM, between 1.4 mM to 5 mM, between 1 mM to 10 mM, between 5 mM to 7.5 mM, at least 0.8 mM, at least 1.6 mM, about 1.1 mM, about 1.5 mM, about 2 mM, about 4 mM, at least 8 mM, about 1.2 mM, about 2.8 mM, about 3 mM, about 6 mM, about 10 mM, about 11 mM or about 12 mM. In one example, the nucleic acid is present in the composition in a concentration of at least 1.6 mM. In another example, the nucleic acid is present in the composition in a concentration of at least 10 mM. In yet another example, the nucleic acid is present in a concentration of about 1.6 mM. In a further example, the nucleic acid is present in a concentration of about 10 mM. In yet another example, the composition comprises two nucleic acids, wherein one nucleic acid is present in a concentration of 1 mM and the other nucleic acid is present in a concentration of 0.16 mM. In another example, one nucleic acid is present in a concentration of 10 mM and the other nucleic acid is present in a concentration of 1.6 mM. In a further example, one nucleic acid is present in a concentration of 100 mM and the other nucleic acid is present in a concentration of 16 mM.

The present disclosure refers to the use of a composition for, for example, the expansion of myoblasts. In one example, the composition as described herein comprises a fibroblast growth factor, a Notch signalling agonist and at least one nucleic acid. In another example, the composition as described herein comprises a fibroblast growth factor, a Notch signalling agonist and at least two nucleic acids. In yet another example, the composition as described herein comprises basic fibroblast growth factor (bFGF), a Notch signalling agonist and at least two nucleic acids. In another example, the composition as described herein comprises a fibroblast growth factor, Delta-like ligand 1 (DLL1) and at least two nucleic acids. In another example, the composition as described herein comprises a fibroblast growth factor, a Notch signalling agonist and at least two nucleic acids, wherein the two nucleic acids are hypoxanthine and thymine. In yet another example, the composition as described herein comprises basic fibroblast growth factor (bFGF), a Notch signalling agonist and at least two nucleic acids, wherein the two nucleic acids are hypoxanthine and thymine. In another example, the composition as described herein comprises a fibroblast growth factor, Delta-like ligand 1 (DLL1) and at least two nucleic acids, wherein the two nucleic acids are hypoxanthine and thymine. In another example, the composition as described herein comprises basic fibroblast growth factor (bFGF), Delta-like ligand 1 (DLL1) and at least two nucleic acids, wherein the two nucleic acids are hypoxanthine and thymine.

In one example, the composition as described herein comprises basic fibroblast growth factor (bFGF) is present in a concentration of about 20 ng/ml, Delta-like ligand 1 (DLL1) is present in a concentration of about 50 μg/ml and at least two nucleic acids, hypoxanthine and thymine are present in a concentration of about 10 mM and 1.6 mM, respectively.

The composition as described herein may optionally comprise one or more of the following components: a Wnt signalling agonist, an adenylyl cyclase, a vitamin and a salt.

As used herein, the term “Mint signalling agonist” refers to a molecule that acts as an agonist for the Wnt signalling pathway. The Wnt signalling pathways in general are known as a group of signal transduction pathways made up of proteins that pass signals into a cell through cell surface receptors. Three Wnt signalling pathways are known: the canonical Wnt pathway, the non-canonical planar cell polarity pathway and the non-canonical Wnt/calcium pathway. All three pathways are activated by binding a Wnt-protein ligand to a Frizzled family receptor, which passes the biological signal to the protein dishevelled inside the cell. The canonical Wnt pathway leads to regulation of gene transcription. The non-canonical planar cell polarity pathway regulates the cytoskeleton that is responsible for the shape of the cell. The non-canonical Wnt/calcium pathway regulates calcium inside the cell. Wnt signalling pathways use either nearby cell-cell communication (paracrine) or same-cell communication (autocrine). These signalling pathways are known in the art to be highly evolutionarily conserved in animals, meaning these pathways are similar across different animal species. Wnt signalling is also known to play a role in carcinogenesis and in embryonic development. An example of a Wnt signalling agonist can be, but is not limited to, 6-[[2-[[4-(2,4-dichlorophenyl)-5-(5-methyl-1H-imidazol-2-yl)-2 pyrimidinyl]amino]ethyl]amino]-3-pyridinecarbonitrile (CHIR99021), lithium salts, 6-Bromoindirubin-3′-oxime (BIO), N2-(2-(4-(2,4-dichlorophenyl)-5-(1H-imidazol-1-yl)pyrimidin-2-ylamino)ethyl)-5-nitropyridine-2,6-diamine (CHIR98014), N-(4-Methoxybenzyl)-N′-(5-nitro-1,3-thiazol-2-yl)urea (ARA014418), (4Z)-4-(2-Amino-4-oxo-1H-imidazol-5-ylidene)-2-bromo-1,5,6,7-tetrahydropyrrolo[2,3-c]azepin-8-one (hymenialdisine), 2-(2-chlorophenyl)-5,7-dihydroxy-8-[(3S,4R)-3-hydroxy-1-methyl-4-piperidinyl]-4-chromenone (flavopiridol), 7-Butyl-6-(4-methoxyphenyl)-[5H]pyrrolo[2,3-b]pyrazine (aloisine), dichlorophenyl)-4-(1-methyl-1H-indol-3-yl)-1H-pyrrole-2,5-dione (SB216763), 3-((3-Chloro-4-hydroxyphenyl)amino)-4-(2-nitrophenyl)-(1H)-pyrrole-2,5-dione (SB415286), 9-bromo-7,12-dihydro-indolo-[3,2-d]-[1]benzazepin-6(5H)-one (kenpaullone) and derivatives thereof.

As used herein, the term “adenylyl cyclase” refers to is an enzyme with key regulatory roles in essentially all cells. It is the most polyphyletic known enzyme: six distinct classes have been described, namely classes I to VI, all catalysing the same reaction but representing unrelated gene families with no known sequence or structural homology. The best known class of adenylyl cyclases is class III or AC-III. AC-III occurs widely in eukaryotes and has important roles in many human tissues. All classes of adenylyl cyclases catalyse the conversion of adenosine triphosphate (ATP) to 3′,5′-cyclic AMP (cAMP) and pyrophosphate. Magnesium ions are generally required and appear to be closely involved in the enzymatic mechanism. The cAMP produced by adenylyl cyclase then serves as a regulatory signal via specific cAMP-binding proteins, either transcription factors, enzymes (e.g., cAMP-dependent kinases), or ion transporters. Thus, in one example, the adenylyl cyclase signalling agonist a labdane diterpene and preferably a forskolin or a derivative thereof. In another example, the adenylyl cyclase signalling agonist can be, but is not limited to, a non-hydrolysable analogue of cAMP, an isoprotenol, a vasoactive intestinal peptide, a calcium ionophore, a membrane depolarization agent, a cAMP stimulatory macrophage-derived factor, a macrophage activating agent, a phosphodiesterase inhibitor, a pituitary adenylate cyclase activating peptide (PACAP), a cholera toxin, a prostaglandin compound, a beta 2-adrenoreceptor agonist, and derivatives thereof.

As used herein, the term “vitamin” refers to an organic compound and a vital nutrient that an organism requires in limited amounts. An organic chemical compound (or related set of compounds) is called a vitamin when the organism cannot synthesize the compound in sufficient quantities, and it must be obtained through the diet. Thus, the term “vitamin” is conditional upon the circumstances and the particular organism. For example, ascorbic acid (one form of vitamin C) is a vitamin for humans, but not for most other animal organisms. By convention the term “vitamin” includes neither other essential nutrients, such as dietary minerals, essential fatty acids, or essential amino acids (which are needed in greater amounts than vitamins), nor the great number of other nutrients that promote health, and are required less often to maintain the health of the organism. Thirteen vitamins are universally recognized at present. These are vitamin A, vitamin B1, vitamin B2, vitamin B3, vitamin B5, vitamin B6, vitamin B7, vitamin B9, vitamin B12, vitamin C, vitamin D, vitamin E and vitamin K. Vitamins are generally classified by their biological and chemical activity, and not by their structure. Thus, each “vitamin” refers to a number of vitamer compounds that all show the biological activity associated with a particular vitamin. For example, a set of chemicals is grouped under an alphabetized vitamin “generic descriptor” title, such as “vitamin A”, which includes the compounds retinal, retinol, and four known carotenoids. Vitamins by definition are convertible to the active form of the vitamin in the body, and are sometimes inter-convertible to one another, as well. Vitamins have diverse biochemical functions. Some, such as vitamin D, have hormone-like functions as regulators of mineral metabolism, or regulators of cell and tissue growth and differentiation (such as some forms of vitamin A). Other vitamins function as antioxidants (e.g., vitamin E and sometimes vitamin C). The largest number of vitamins, the B complex vitamins, function mainly as enzyme cofactors (coenzymes) or the precursors of the same; coenzymes help enzymes in their work as catalysts in metabolism. In this role, vitamins may be tightly bound to enzymes as part of prosthetic groups. For example, biotin is part of enzymes involved in making fatty acids. Vitamins may also be less tightly bound to enzyme catalysts as coenzymes, detachable molecules that function to carry chemical groups or electrons between molecules. For example, folic acid may carry methyl, formyl, and methylene groups in the cell. Although these roles in assisting enzyme-substrate reactions are vitamins' best-known function, the other vitamin functions are equally important. Thus, in example, the vitamin can be a B-complex vitamin. In another example, the vitamin can be vitamin B12 or cobalamin.

Methods of using the claimed compositions are also described herein. Thus, in one example, the present disclosure describes a method for preparing myofibers or myotubes, comprising contacting a skeletal muscle stem cell or progenitor cell with the composition as described herein. In another example, the present disclosure describes a method for inducing expansion of skeletal muscle progenitor cells comprising the step of contacting a skeletal muscle stem cell or progenitor cell with the composition as described herein.

Other uses of the compositions described herein include addressing the degeneration of muscle mass during, for example, but not limited to cachexia as a result of, for example, rapid loss of muscle in all cancers, chronic kidney disease, chronic obstructive pulmonary disease (COPD), acquired immune deficiency syndrome (AIDS) or other chronic diseases, sarcopenia (aging-associated muscle wasting), muscle atrophy, muscle dystrophy and diabetes.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXPERIMENTAL SECTION

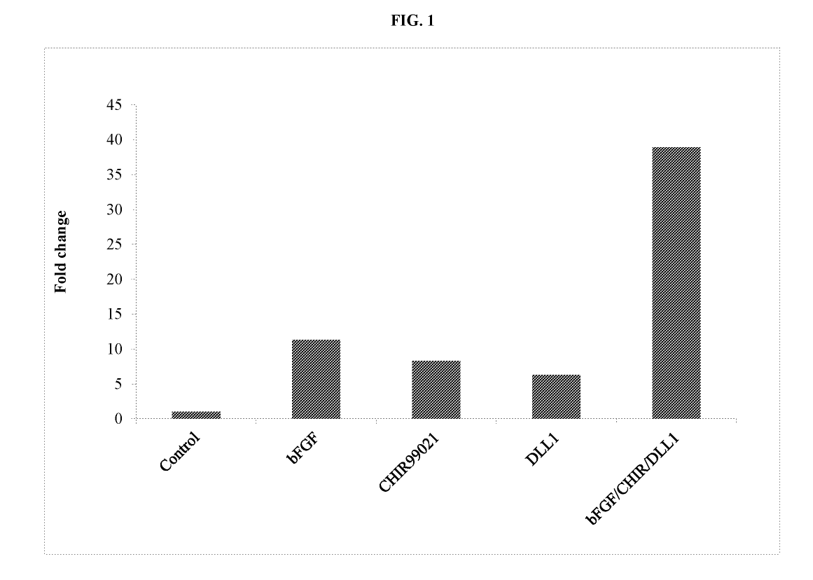

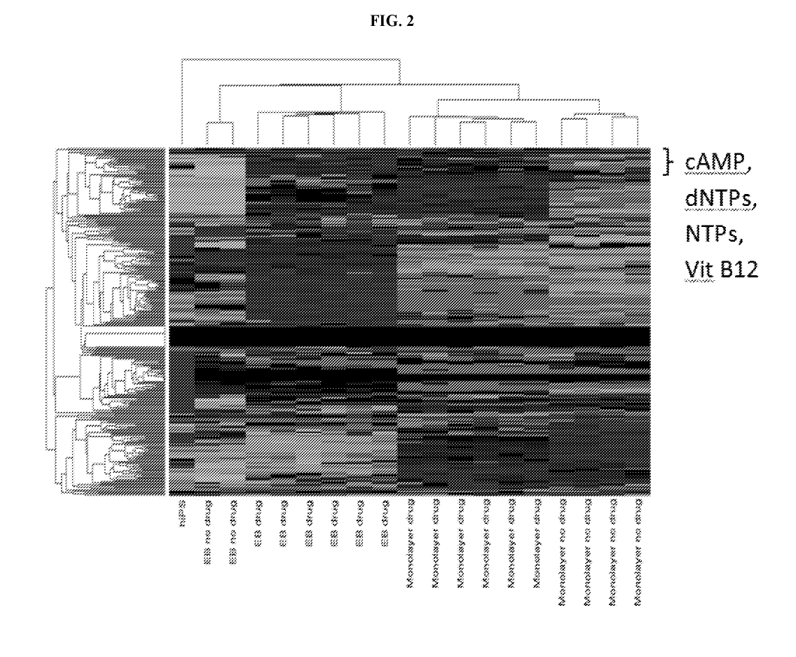

By mimicking and optimizing the embryonic signalling pathways invoked during myogenesis (FIG. 1), it was identified that the Wnt/β-catenin agonist CHIR99021 (3 μM), the FGF agonist bFGF (20 ng/ml), and the Notch agonist DLL1 (2.5 μg/ml), added to standard DMEM media (20% FBS, 1% Pen-Strep), could specify human embryonic stem cells/induced pluripotent stem cells to undergo myogenesis over 45 days to convert into Pax7+ myoblasts. This combination could only specify the transient conversion of human embryonic stem cells/induced pluripotent stem cells to myoblasts, but was not able to induce large-scale expansion of Pax7⁺ myoblasts. In order to elicit the metabolic conditions necessary for myoblast expansion, LC-MS metabolomics was performed on human induced pluripotent stem cells, relative to myoblast-enriched embryoid bodies (EBs), and monolayers of differentiated myotubes, with or without the drug treatment (FIG. 2).

It was found that metabolites were specifically increased in human myoblasts, thus showing the metabolic fluxes required by human myoblasts. These include an increase in cyclic AMP (cAMP), nucleotides (dNTPs and NTPs), and cobalamin (vitamin B12).

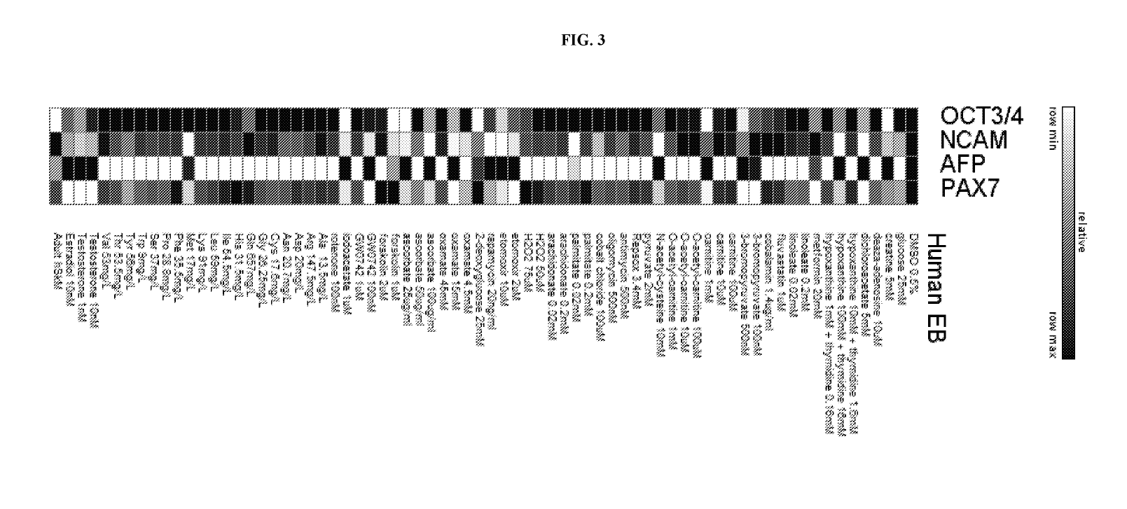

Thus, the adenylyl cyclase agonist Forskolin was tested to induce cAMP synthesis, and cobalamin was administered to see if these compounds boosted the expansion of human myoblasts. It was also tested whether glutamine, hypoxanthine and thymidine—all of which are rate-limiting for nucleotide synthesis—could boost human myoblast expansion. Against this background, a variety of other small molecule drugs and metabolites were screened, including amino acids and fatty acids, thereby testing their effects on myoblast expansion. A real-time polymerase chain reaction (qPCR) screen was then performed for various lineage markers (pluripotency marker OCT3/4, neuromuscular marker NCAM, endoderm marker AFP, muscle marker Pax7) on human induced pluripotent stem cell-derived myoblasts exposed to these 70 conditions (FIG. 3).

The results show that forskolin, cobalamin, glutamine, hypoxanthine and thymidine, each individually, could specifically increase Pax7 expression during FGF/Wnt/Notch-induced myogenesis from human induced pluripotent stem cells. These results indicate that a cocktail of forskolin, cobalamin, glutamine, hypoxanthine and thymidine could boost myoblast expansion after specification by FGF/Wnt/Notch.

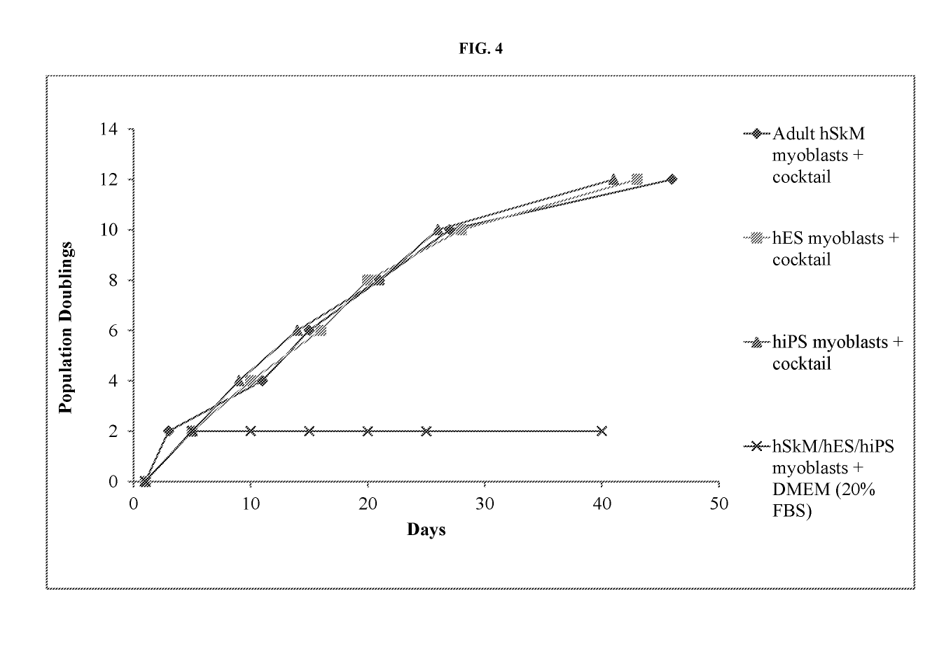

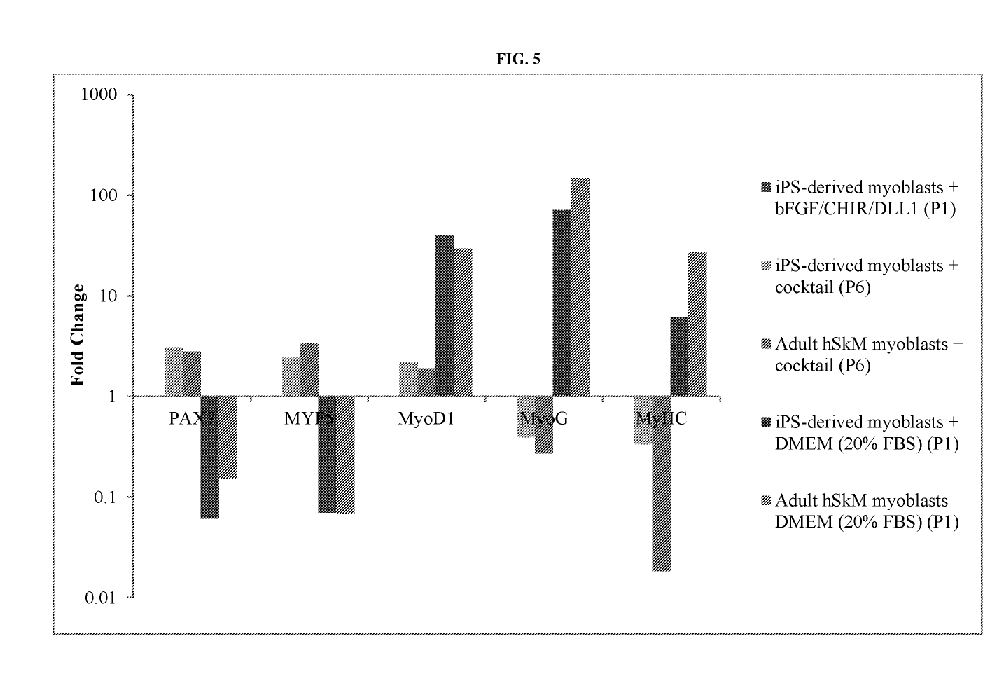

To validate this hypothesis, human embryonic stem cells/induced pluripotent stem cell-derived myoblasts were cultured in a cocktail of DMEM media (20% FBS, 1% Pen-Strep) supplemented with basic fibroblast growth factor (bFGF), CHIR99021, DLL1, forskolin, cobalamin, glutamine, hypoxanthine and thymidine. Human embryonic stem cells, as used herein, were cultured to passage 31 from the WA01 cell-line manufactured and sold by WiCell Research Institute Inc (Madison, Wis.). Induced pluripotent stem cells were cultured to passage 56 from the BJ1-iPSC cell-line generated in-house. It was possible to expand human embryonic stem cell/induced pluripotent stem cell-derived myoblasts in this cocktail for at least 6 passages, with each passage being a split in the ratio of 1:4 in each well of a 6-well plate (seeded 60,000 cells, per well, per passage. This translates into an over 2¹²-fold expansion of the initial 60,000 cells, or >4000-fold expansion (FIG. 4). Interestingly, when the same treatment was performed on human primary adult myoblasts (hSkM), a similar expansion of human primary adult myoblasts was observed—indicating that the described composition/cocktail could expand both human embryonic stem cells/human induced pluripotent stem cell-derived and primary adult myoblasts (FIG. 4). Gene expression profiling proved that these myoblasts remained Pax7⁺; MYF5⁺ after 6 passages in culture (FIG. 5). In contrast, DMEM (20% FBS, 1% Pen-Strep) failed to expand the myoblasts, as they rapidly differentiated into MyoG⁺; MyHC⁺ myotubes (FIGS. 4 and 5).

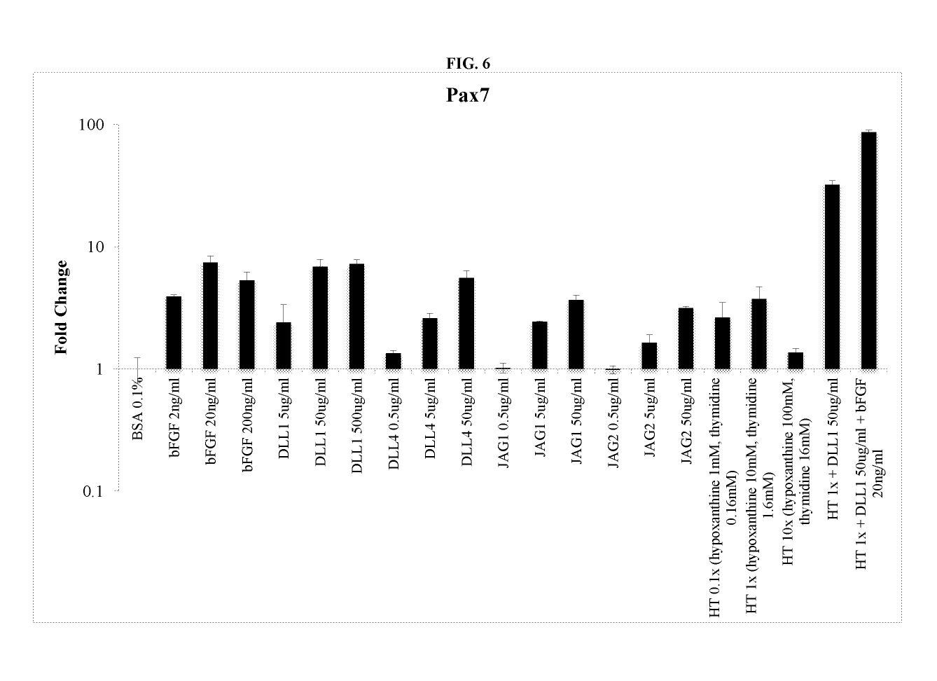

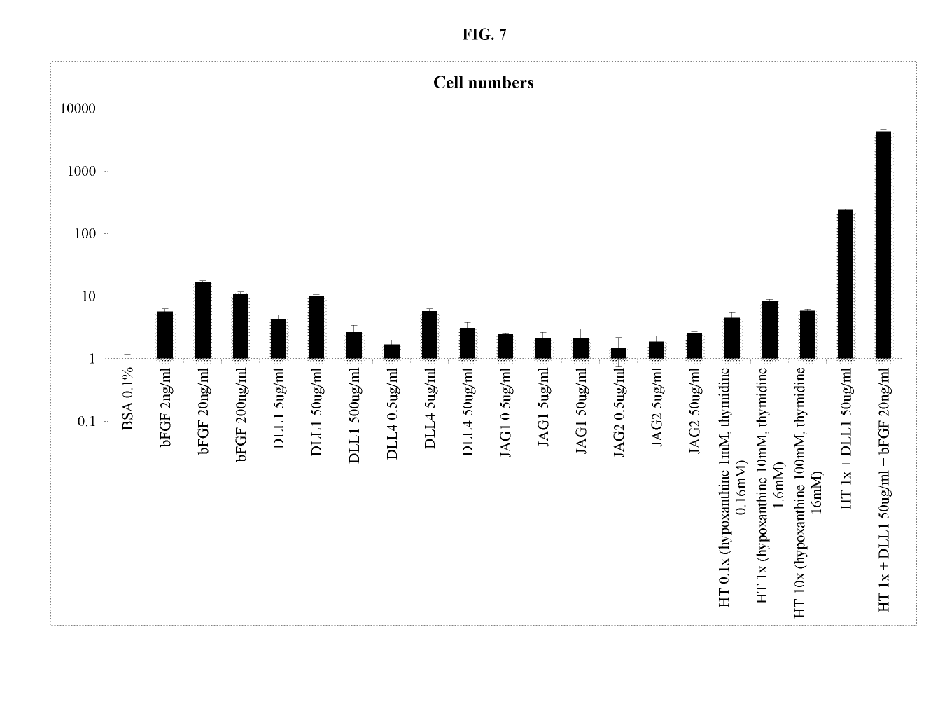

With further optimization of various constituents at different concentrations (FIG. 6), an optimal composition comprising FGF2 (20 ng/ml), DLL1 (50 ug/ml), and HT supplement (hypoxanthine 10 mM, thymidine 1.6 mM), was achieved, thereby eliminating other components from the composition. This optimized composition produces even higher levels of the myoblast marker Pax7, nearly a hundred-fold (FIG. 6). It also expands the myoblast numbers by over 4000-fold (FIG. 7).

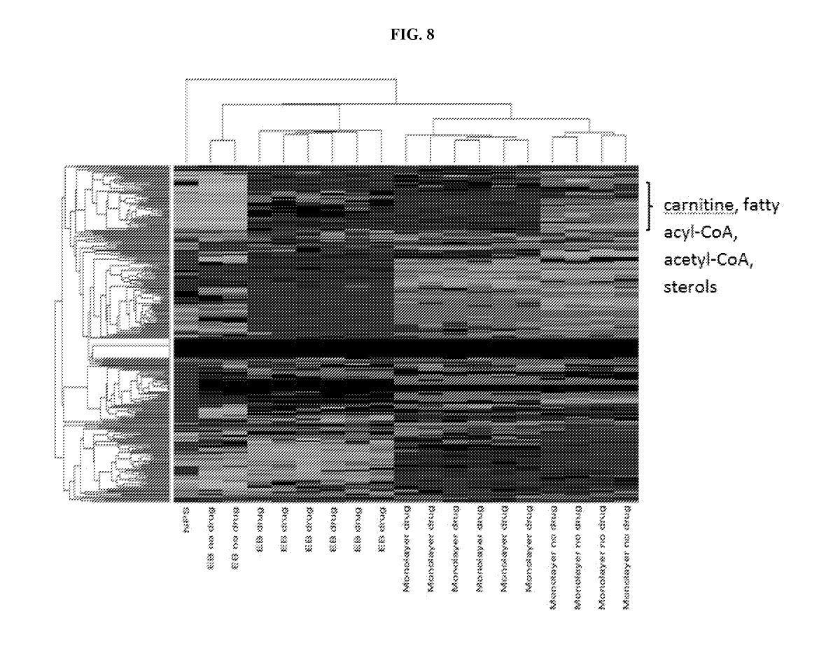

In order to elucidate the metabolic conditions necessary to enhance myotube differentiation, liquid chromatography-mass spectrometry metabolomics were performed on human induced pluripotent stem cells, relative to myoblast embryoid bodies (EBs), and monolayers of differentiated cells, with or without myogenic drug treatment (FIG. 8). It was found that metabolites were specifically increased in human myotubes, thus implying the metabolic fluxes required by human myotubes. These metabolites included an increase in carnitine, fatty acyl-CoA, acetyl-CoA and sterols.

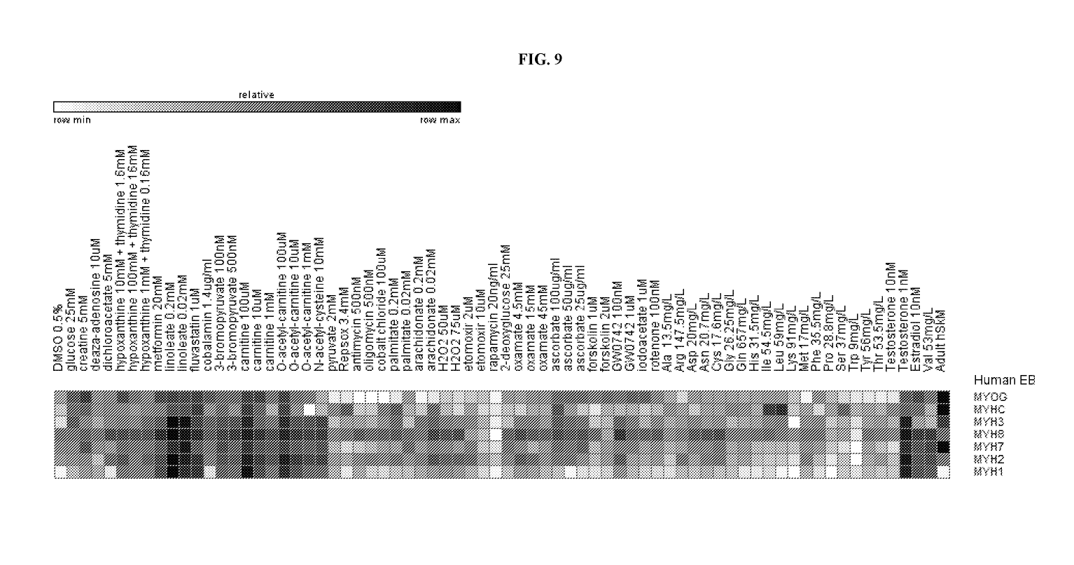

Thus carnitine to facilitate fatty acid oxidation, the fatty acids linoleate, arachidonate and palmitate to fuel fatty acid oxidation were tested. It was also tested whether statin inhibitors of steroid synthesis, such as fluvastatin, and steroids like testosterone or progesterone could boost human myotube differentiation. Against this background, a variety of other small molecule drugs and metabolites were also screened, including OxPhos inhibitors and vitamins, to test their effects on myotube differentiation. A qPCR screen was then performed for various myogenic differentiation markers on human induced pluripotent stem cell-derived myoblasts exposed to these 70 conditions (FIG. 9). These results showed that carnitine, O-acetyl-carnitine, linoleate, fluvastatin, and testosterone each individually could specifically increase MyoD, MyoG and MYHC (myosin heavy chain) expression during drug-induced myogenesis from human induced pluripotent stem cells. The results also suggested that a cocktail of carnitine/O-acetyl-carnitine, linoleate, fluvastatin, and testosterone could boost myotube differentiation.

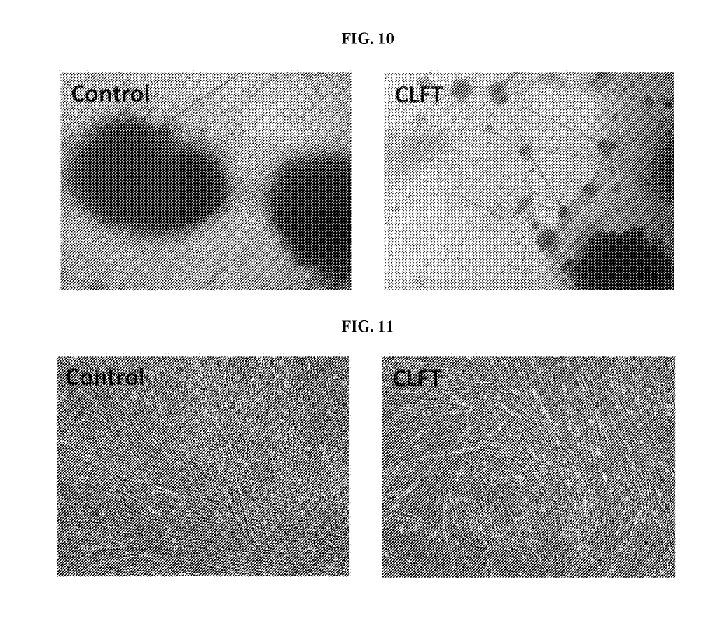

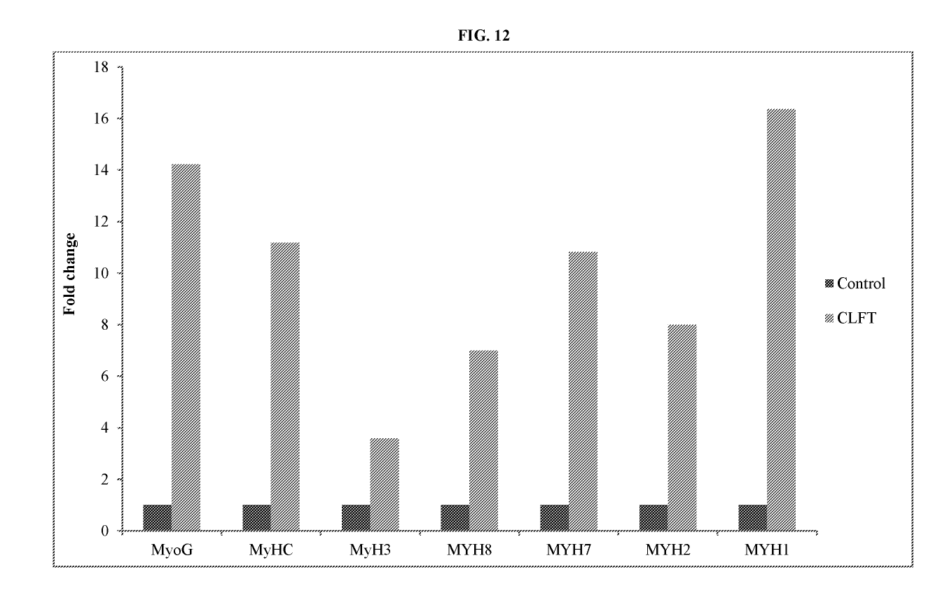

To validate this hypothesis, induced pluripotent stem cell-derived myoblasts were cultured either in the standard differentiation media (DMEM 2% horse serum, 1% Pen-Strep; “control”), or supplemented it with a cocktail of carnitine, linoleate, fluvastatin and testosterone (CLFT). The CLFT composition could accelerate and enhance the differentiation of human embryonic stem cell/induced pluripotent stem cell-derived myoblasts into myotubes (FIG. 10). Interestingly, when the same treatment was performed on human primary adult myoblasts, a similar enhancement of the myogenic differentiation of human primary adult myoblasts into myotubes was observed—indicating that our CLFT supplement could enhance differentiation of both embryonic stem cell/induced pluripotent stem cell-derived and primary adult myoblasts (FIG. 11). Gene expression profiling for various myotube markers such as MyoG and MyHC proved that the CLFT composition was able to enhance many myogenic differentiation markers more than 10-fold (>10-fold), relative to control media (FIG. 12).

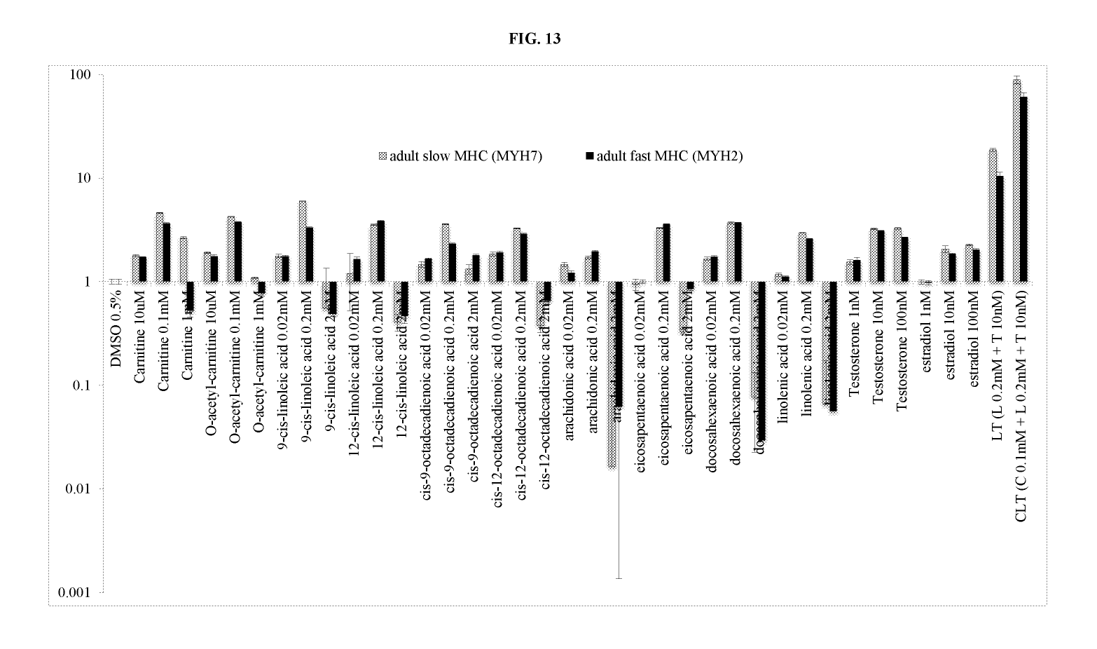

With further optimization of various versions of fatty acids at different concentrations (FIG. 13), an optimal composition comprising of L-carnitine (0.1 mM), 9-cis-linoleic acid (CLA; 0.2 mM), and dihydrotestosterone (DHT; 10 nM), was established, thereby eliminating fluvastatin from the mixture. This composition produces even higher levels of adult slow-twitch and fast-twitch myosin heavy chain markers (MYH7 and MYH2 respectively), nearly a hundred-fold, indicating that differentiation of the myoblast cells into myotubes had been achieved.

1-38. (canceled)

39: A composition for preparing a myofiber or myotube from a skeletal muscle stem cell or progenitor cell comprising a carnitine and/or a derivative thereof, a fatty add and a steroid.

40: The composition of claim 39, wherein the skeletal muscle progenitor cell is a myoblast cell; and/or wherein the myoblast cell is a primary myoblast cell.

41: The composition of claim 39, wherein the skeletal muscle stem cell is a myosatellite cell.

42: The composition of claim 40, wherein the myoblast cell is derived from an embryonic stem (ES) cell, an induced pluripotent stem (IPS) cell, a mesenchymal stem cell, a neural stem cell or a multipotent stem cell; and/or wherein the myoblast cell expresses high levels of Pax7 mRNA as compared to Pax7 mRNA expression in a human embryonic stem cell.

43: The composition of claim 39, wherein the skeletal muscle stem cell or progenitor cell is derived from a mammal; and/or wherein the skeletal muscle stem cell or progenitor cell is derived from a human, rodent or primate.

44: The composition of claim 39, wherein the carnitine or derivative thereof is L-carnitine or an acyl carnitine; and/or wherein the acyl carnitine is O-acetyl-carnitine, O-propionyl-carnitine or O-butanoyl-carnitine.

45: The composition of claim 39, wherein the fatty add is an unsaturated fatty add; and/or wherein the fatty acid is an omega 3 or an omega 6 fatty acid.

46: The composition of claim 45, wherein the fatty add is selected from a group consisting of a linoleic acid, an arachidonic add, an eicosapentaenoic add, a docosahexaenoic acid, a linolenic acid and derivatives of the aforementioned fatty adds; and/or wherein the linoleic acid is selected from a group consisting of 9-cis-linoleic acid, 12-cis-linoleic acid, cis-9 octadecadienoic acid and cis-12-octadecadienoic add.

47: The composition of claim 39, wherein the steroid is a sex steroid; and/or wherein the sex steroid is selected from a group consisting of an androgen and estrogen; and/or wherein the steroid is a testosterone, estradiol or a derivative thereof.

48: The composition of claim 39, wherein the composition comprises L-carnitine, linoleic acid and testosterone; and/or wherein the composition comprises L-carnitine in a concentration of about 0.1 mM, linoleic add in a concentration of about 0.2 mM and testosterone in a concentration of about 10 mM.

49: A composition for inducing expansion of skeletal muscle stem cells or progenitor cells comprising a fibroblast growth factor signalling agonist, a Notch signalling agonist and a nucleic add.

50: The composition of claim 49, wherein the skeletal muscle progenitor cell is a myoblast cell, and/or wherein the myoblast cell is a primary myoblast cell.

51: The composition of claim 49, wherein the skeletal muscle stem cell is a myosatellite cell.

52: The composition of claim 50, wherein the myoblast cell is derived from an embryonic stem (ES) cell, an induced pluripotent stem (IPS) cell, a mesenchymal stem cell, a neural stem cell, or a multipotent stem cell; and/or wherein the myoblast cell expresses high levels of Pax7 mRNA as compared to Pax7 mRNA expression in a human embryonic stem cell.

53: The composition of claim 49, wherein the skeletal muscle stem cell or progenitor cell is derived from a mammal; and/or wherein the skeletal muscle stem cell or progenitor cell is derived from a human, rodent or primate.

54: The composition of claim 49, wherein the fibroblast growth factor signalling agonist is a fibroblast growth factor (FGF) and/or wherein the fibroblast growth factor signalling agonist is selected from a group consisting of FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22 and FGF23; and/or wherein the fibroblast growth factor signalling receptor agonist is FGF2 (basic fibroblast growth factor; bFGF) (bFGF) or a derivative thereof.

55: The composition of claim 49, wherein the Notch signalling agonist is a Delta-like ligand (DLL), a Jagged/Serrate ligand or a derivative thereof; and/or wherein the Delta-like ligand (DLL) is selected from a group consisting of Delta-like 1 (DLL1), Delta-like 3 (DLL3) and Delta-like 4 (DLL4); and/or wherein the Jagged/Serrate ligand is selected from a group consisting of Jagged 1 (JAG1), Jagged 2 (JAG2) and Serrate.

56: The composition of claim 49, wherein the nucleic add is derived from a nitrogenous base selected from a group consisting of hypoxanthine, adenine, guanine, thymine, cytosine, inosine, xanthine and a derivative of the aforementioned nitrogenous bases.

57: The composition of claim 49, wherein the composition comprises basic fibroblast growth factor (bFGF), Delta-like ligand 1 (DLL1), hypoxanthine and thymine; and/or wherein the composition comprises basic fibroblast growth factor (bFGF) in a concentration of about 20 ng/ml, Delta-like ligand 1 (DLL1) in a concentration of about 50 μg/ml, hypoxanthine in a concentration of about 10 mM and thymine in a concentration of about 1.6 mM.

58: A method for preparing myofibers or myotubes, comprising contacting a skeletal muscle stem cell or progenitor cell with a composition for preparing a myofiber or myotube from a skeletal muscle stem cell or progenitor cell comprising a carnitine and/or a derivative thereof, a fatty add and a steroid.

59: A method for inducing expansion of skeletal muscle progenitor cells comprising contacting a skeletal muscle stem cell or progenitor cell with a composition for inducing expansion of skeletal muscle stem cells or progenitor cells comprising a fibroblast growth factor signalling agonist, a Notch signalling agonist and a nucleic add.
